(19) United States
(12) Patent Application Publication
(10) Pub. No.: US 2023/0181007 A1
(43) Pub. Date: Jun. 15, 2023

(54) METHOD OF MOLDING ENDOSCOPE COMPONENT, AND ENDOSCOPE

(71) Applicant: FUJIFILM Corporation, Tokyo (JP)

(72) Inventors: Takashi HARADA, Ashigarakami-gun (JP); Kimitake FUKUSHIMA, Ashigarakami-gun (JP)

(73) Assignee: FUJIFILM Corporation, Tokyo (JP)

(21) Appl. No.: 18/168,860

(22) Filed: Feb. 14, 2023

Related U.S. Application Data
(63) Continuation of application No. PCT/JP2021/031911, filed on Aug. 31, 2021.

(30) Foreign Application Priority Data
Sep. 4, 2020 (JP) 2020-148708

(51) Int. Cl. A61B 1/00

(52) U.S. Cl. CPC A61B 1/0011; A61B 1/00098

(57) ABSTRACT

A method of molding an endoscope component has a step of forming a covering portion (150), in which a gap filling member (152) that fills a gap formed between a plurality of elemental wires (40a) is disposed, at one end portion of a stranded wire (40); a step of inserting and disposing the one end portion of the stranded wire (40) into a cavity (106) of a mold (100) such that the covering portion (150) is disposed at a boundary portion between an inner side and an outer side of the endoscope component; a step of injecting a formation material (108), which is a material of the endoscope component, into the cavity (106) and filling the cavity (106) with the formation material (108) to integrally mold the endoscope component and the stranded wire (40); and a step of removing the endoscope component integrally molded with the stranded wire (40) from the mold (100).

### CROSS-REFERENCE TO RELATED APPLICATIONS

The present application is a Continuation of PCT International Application No. PCT/JP2021/031911 filed on Aug. 31, 2021 claiming priority under 35 U.S.C § 119(a) to Japanese Patent Application No. 2020-148708 filed on Sep. 4, 2020. Each of the above applications is hereby expressly incorporated by reference, in its entirety, into the present application.

### BACKGROUND OF THE INVENTION

### 1. Field of the Invention

The present invention relates to a method of molding an endoscope component and to an endoscope, and, particularly, to a method of molding an endoscope component and to an endoscope, in which a wire and the endoscope component are directly connected to each other.

### 2. Description of the Related Art

In an endoscope, various treatment tools are introduced from a treatment-tool introduction port provided at an operating unit, and the treatment tools are led out to the outside from a treatment-tool lead-out port that opens in a distal end portion of an insertion unit, and are used for treatment. For example, a treatment tool such as a guide wire or a contrast tube is used in a duodenoscope. In such a treatment tool, it is necessary to change a lead-out direction at the distal end portion in order to treat a desired location in a subject. For this reason, the distal end portion of the insertion unit is provided with a raising base that changes the lead-out direction of the treatment tool. In addition, the endoscope is provided with a treatment-tool raising mechanism that changes the posture of the raising base between a raised position and a lowered position.

As a treatment-tool raising mechanism, a lever type (close type) mechanism is known in which a raising-base housing chamber that houses the raising base and a lever housing chamber that houses a lever are disposed adjacent to each other via a partition wall at the distal end portion of the insertion unit. Further, there is known a wire traction type (open type) mechanism in which a distal end of a traction wire is directly attached to the raising base. As a method of directly attaching a distal end of an operating wire to the raising base, a method of attaching the raising base and the operating wire by brazing, welding, or caulking is performed.

None of these methods are suitable for disposal because the assembly is complicated or the number of steps is large and the cost is high.

As a method of molding the raising base, JP1994-315459A (JP-H06-315459A) describes that a forceps standing wire and a forceps standing base are integrally formed.

### SUMMARY OF THE INVENTION

If the raising base and the operating wire can be directly and integrally molded by insert molding, it is possible to simplify the raising base and drastically reduce man-hours, and to make the raising base a disposable component. Usually, in the case of insert molding, a formation material is prevented from flowing out of a cavity of a mold by adjusting the pressing force of the mold at the time of the molding. There is concern that, when the formation material flows out of the cavity, the formation material is solidified on the surface of the operating wire, and the solidified formation material falls off due to the operation of the operating wire. In consideration of tensile strength and flexibility, it is preferable to use a stranded wire as the operating wire. However, when the stranded wire is used as the operating wire, as a result of increasing the pressing force in order to prevent the formation material from flowing out, the stranded wire is loosened, and thus the outflow cannot be prevented.

JP1994-315459A (JP-H06-315459A) does not consider anything about the existence of the above-mentioned problem when the forceps standing base (raising base) and the forceps standing wire (operating wire) are integrally molded, and does not disclose or suggest a specific countermeasure therefor.

The present invention has been made in view of such circumstances, and an object of the present invention is to provide a method of molding an endoscope component and an endoscope, capable of preventing a formation material of the endoscope component from flowing out of a gap between elemental wires of a stranded wire when the endoscope component is integrally molded with the stranded wire.

To this end of the present invention, a method of molding an endoscope component according to the present invention is a method of molding an endoscope component, in which the endoscope component constituting an endoscope is integrally molded with a stranded wire composed of a plurality of elemental wires, the method having a step of forming a covering portion, in which a gap filling member that fills a gap formed between the plurality of elemental wires is disposed, at one end portion of the stranded wire; a step of inserting and disposing the one end portion of the stranded wire into a cavity of a mold such that the covering portion is disposed at a boundary portion between an inner side and an outer side of the endoscope component; a step of injecting a formation material, which is a material of the endoscope component, into the cavity and filling the cavity with the formation material to integrally mold the endoscope component and the stranded wire; and a step of removing the endoscope component integrally molded with the stranded wire from the mold.

In one form of the present invention, it is preferable that, in the inserting and disposing step, a part of the covering portion be disposed on an outer side of the cavity.

In one form of the present invention, it is preferable that the endoscope component be formed by injection molding of a resin.

In one form of the present invention, it is preferable that the endoscope component be formed by metal injection molding of metal powder.

In one form of the present invention, it is preferable that, in the step of forming the covering portion, the gap of the stranded wire be filled by applying an adhesive having a melting point higher than a melting point of the formation material to the stranded wire.

In one form of the present invention, it is preferable that, in the step of forming the covering portion, the gap of the stranded wire be filled by coating the stranded wire with a resin having a melting point higher than a melting point of the formation material.

In one form of the present invention, it is preferable that, in the step of forming the covering portion, the gap of the stranded wire be filled by applying solder having a melting point higher than a melting point of the formation material to the stranded wire.

In one form of the present invention, it is preferable that, in the step of forming the covering portion, the gap of the stranded wire be filled by applying a brazing material having a melting point higher than a melting point of the formation material to the stranded wire.

In one form of the present invention, it is preferable that, in the step of forming the covering portion, the gap of the stranded wire be filled by subjecting a pipe into which the stranded wire has been inserted to plastic working.

In one form of the present invention, it is preferable that the endoscope component be a raising base disposed at a distal-end-portion main body provided on a distal end side of an insertion unit of the endoscope.

To this end of the present invention, an endoscope according to the present invention is an endoscope including an endoscope component. The endoscope component is an integrally molded body integrally molded with a stranded wire composed of a plurality of elemental wires. The stranded wire has a covering portion, in which a gap filling member that fills a gap formed between the plurality of elemental wires is disposed, at one end portion of the stranded wire. The covering portion is disposed at a draw-out portion of the stranded wire inside the endoscope component.

In one form of the present invention, it is preferable that the covering portion include an exposed portion exposed from the endoscope component.

In one form of the present invention, it is preferable that the stranded wire include an extending portion on a distal end side of the stranded wire, the extending portion being exposed from the covering portion in the endoscope component.

In one form of the present invention, it is preferable that the stranded wire have a bent portion that is bent on a distal end side of the stranded wire in the endoscope component.

In one form of the present invention, it is preferable that the endoscope component be an injection molded body made of a resin material.

In one form of the present invention, it is preferable that the endoscope component be a metal injection molded body made of a metal material.

In one form of the present invention, it is preferable that the gap filling member be an adhesive having a melting point higher than a melting point of a material constituting the endoscope component, and be provided in the gap of the stranded wire.

In one form of the present invention, it is preferable that the gap filling member be a resin having a melting point higher than a melting point of a material constituting the endoscope component, and be provided in the gap of the stranded wire.

In one form of the present invention, it is preferable that the gap filling member be solder having a melting point higher than a melting point of a material constituting the endoscope component, and be provided in the gap of the stranded wire.

In one form of the present invention, it is preferable that the gap filling member be a brazing material having a melting point higher than a melting point of a material constituting the endoscope component, and be provided in the gap of the stranded wire.

In one form of the present invention, it is preferable that the gap filling member be a pipe externally fitted to an outer peripheral surface of the stranded wire.

In one form of the present invention, it is preferable that the covering portion have a stepped portion on a distal end side of the stranded wire, the stepped portion having an outer diameter larger than an outer diameter of a proximal end side.

In one form of the present invention, it is preferable that the endoscope component be a raising base disposed at a distal-end-portion main body provided on a distal end side of an insertion unit of the endoscope.

According to the present invention, it is possible to prevent a formation material from flowing out of a mold when integrally molding an endoscope component.

### BRIEF DESCRIPTION OF THE DRAWINGS

FIG. 1 is a configuration diagram of an endoscope system including an endoscope;

FIG. 2 is an enlarged perspective view of a distal end portion of the endoscope;

FIG. 3 is a perspective view of a distal-end-portion main body shown in FIG. 2;

FIG. 4 is a perspective view of a cap shown in FIG. 2;

FIG. 5 is a diagram illustrating a process of forming a covering portion on a stranded wire;

FIG. 6 is a diagram illustrating a method of molding a raising base;

FIG. 7 is a diagram illustrating the method of molding the raising base;

FIG. 8 is a diagram illustrating the method of molding the raising base;

FIG. 9 is a perspective view of the raising base;

FIG. 10 is a side view of the raising base;

FIG. 11 is a diagram showing a modification of a raising base;

FIG. 12 is a diagram showing another modification of a raising base;

FIG. 13 is a diagram showing still another modification of a raising base;

FIG. 14 is a diagram showing a different example of a gap filling member;

FIG. 15 is a diagram showing another embodiment of a raising base;

FIG. 16 is a diagram showing still another embodiment of a raising base; and

FIG. 17 is a diagram showing still another embodiment of a raising base.

### DESCRIPTION OF THE PREFERRED EMBODIMENTS

Hereunder, a method of molding an endoscope component and an endoscope according to the present invention will be described with reference to the accompanying drawings. Note that, hereunder, although a raising base disposed at a distal-end-portion main body provided on a distal end side of an insertion unit of the endoscope will be described as an example of an endoscope component, the present invention is not limited thereto.

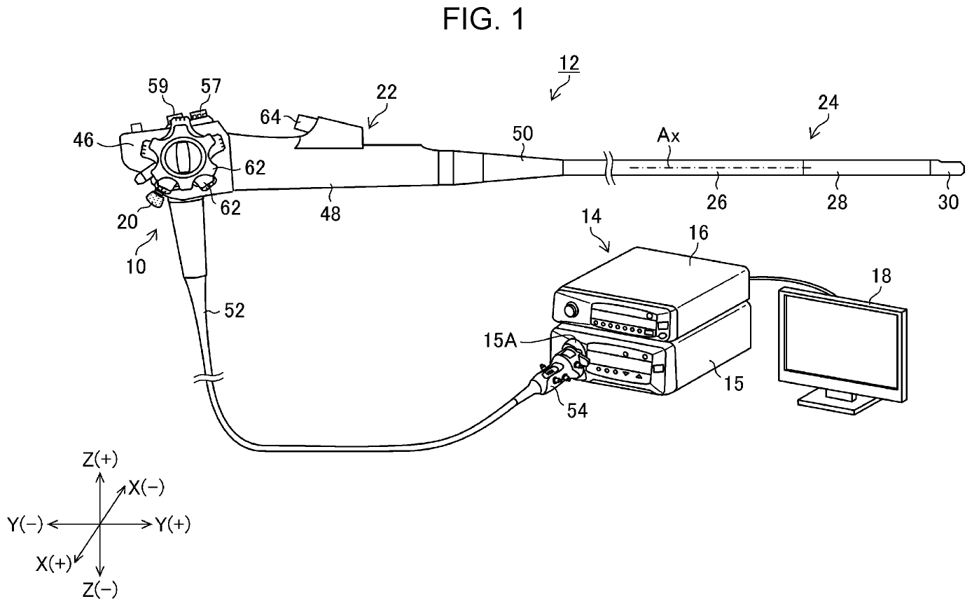

FIG. 1 is a configuration diagram of an endoscope system including a raising base molded by a method of molding an endoscope component of the present invention. An endoscope system 12 includes an endoscope 10, an endoscope processor device 14, and a display 18.

The endoscope 10 includes an operating unit 22 provided with a raising operating lever 20, and an insertion unit 24 provided on a distal end side of the operating unit 22 and inserted into a subject.

The insertion unit 24 has a longitudinal axis Ax from a proximal end toward a distal end, and includes a flexible portion 26, a bending portion 28, and a distal end portion 30 in order from the proximal end toward the distal end. Although a detailed structure of the distal end portion 30 will be described later, first, a schematic structure of the distal end portion 30 will be described.

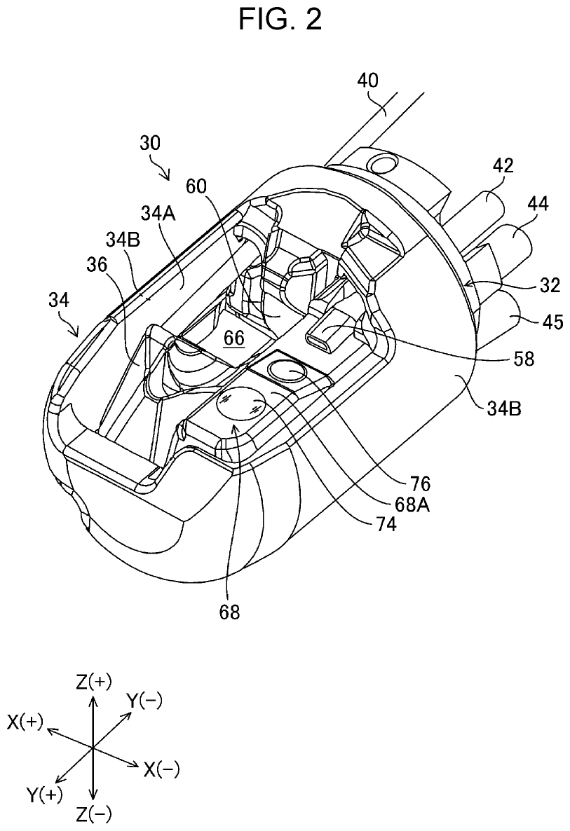

FIG. 2 is an enlarged perspective view of the distal end portion 30. Here, the endoscope 10 (see FIG. 1) of the embodiment is, for example, a side-viewing endoscope used as a duodenoscope, and the distal end portion 30 in FIG. 2 has a structure in the side-viewing endoscope.

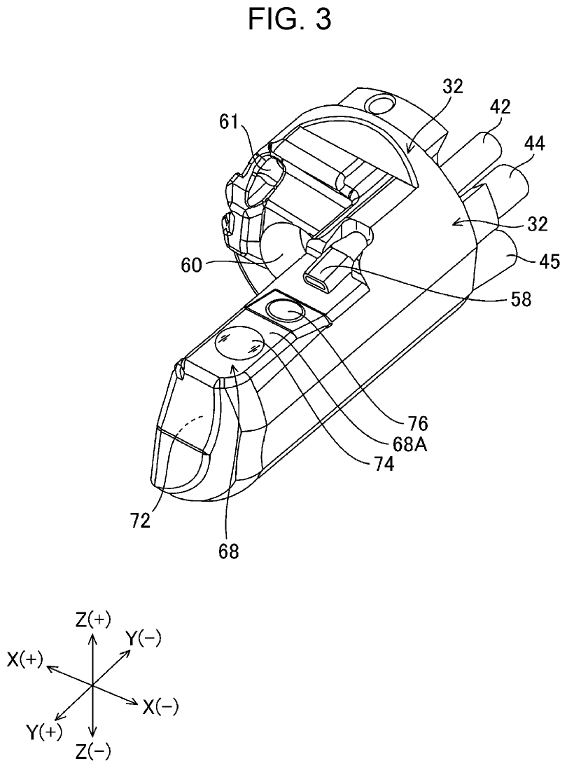

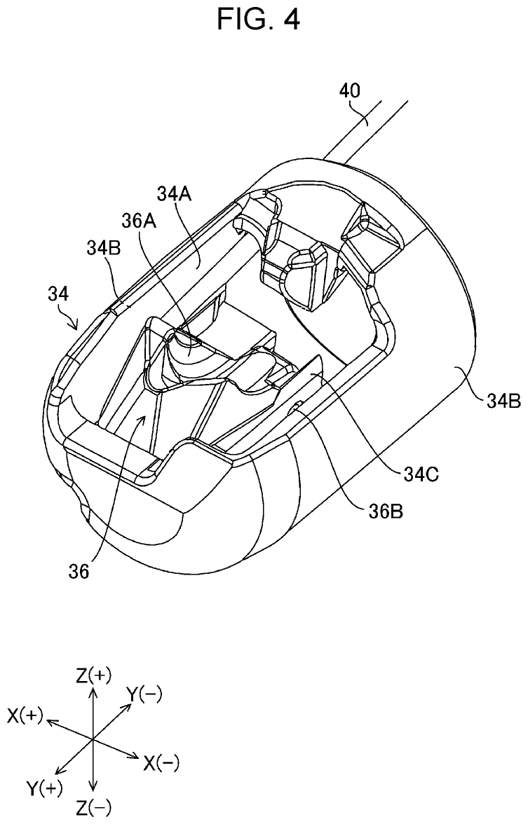

FIG. 3 is a perspective view of a distal-end-portion main body 32 constituting the distal end portion 30. FIG. 4 is a perspective view of a cap 34 constituting the distal end portion 30. As shown in FIG. 2, the distal end portion 30 has the distal-end-portion main body 32 and the cap 34. The cap 34 is attachably and detachably mounted on the distal-end-portion main body 32. The distal-end-portion main body 32 is provided on the distal end side of the insertion unit 24 (see FIG. 1). The distal-end-portion main body 32 is provided with a raising base 36 having a treatment-tool guide surface 36A described later. FIGS. 2 and 4 show a state in which the raising base 36 is positioned in a lowered position.

FIG. 2 also shows various contents disposed inside the insertion unit 24 of the endoscope 10 (see FIG. 1). Specifically, there are provided the raising base 36 for performing an operation of changing a lead-out direction of a distal end portion of a treatment tool (not shown), in which the distal end portion of the treatment tool is led out from the distal-end-portion main body 32, a stranded wire 40 that is an operating wire, an air/water supply tube 42, a cable insertion channel 44 into which a signal cable that transmits an image signal is inserted, and a light-guide insertion channel 45 into which a light guide that transmits illumination light is inserted. The stranded wire 40 is directly connected to the raising base 36, and is connected to the raising base 36 as an integrally molded body in which the raising base 36 and the stranded wire 40 are integrally molded when the raising base 36 is molded. Although not shown in FIG. 2, a treatment instrument channel that guides to the distal-end-portion main body 32 and contents such as an angle wire for performing an operation of changing a bending direction of the bending portion 28 (see FIG. 1) are also provided.

Note that, in the present specification, a three-dimensional orthogonal coordinate system for three axis directions (an X-axis direction, a Y-axis direction, and a Z-axis direction) is used for description. That is, when the distal end portion 30 is viewed from the operating unit 22 and a direction in which a treatment tool (not shown) is led out by the raising base 36 is defined as an upward direction, the upward direction is defined as a Z(+) direction, and a downward direction which is an opposite direction thereto is defined as a Z(−) direction. Further, a rightward direction at that time is defined as an X(+) direction, and a leftward direction is defined as an X(−) direction. In addition, a forward direction (a direction toward the distal end side in a longitudinal-axis-Ax direction of the insertion unit 24) at that time is defined as a Y(+) direction, and a rearward direction (a direction toward a proximal end side in the longitudinal—axis-Ax direction of the insertion unit 24) is defined as a Y(−) direction. Note that the Y-axis direction including the Y(+) direction and the Y(−) direction is parallel to the direction of the longitudinal axis Ax of the insertion unit 24. The Z-axis direction is a direction orthogonal to the direction of the longitudinal axis Ax. The X-axis direction is a direction orthogonal to the Z-axis direction.

Returning to FIG. 1, the operating unit 22 is formed in a substantially cylindrical shape as a whole. The operating unit 22 has an operating-unit main body 46 where the raising operating lever 20 is rotatably provided, and a grasping portion 48 connected to the operating-unit main body 46. A proximal end portion of the insertion unit 24 is provided on a distal end side of the grasping portion 48 via a bend prevention tube 50. The grasping portion 48 is a portion to be grasped by an operator when operating the endoscope 10.

Further, the operating-unit main body 46 includes a universal cable 52. A connector device 54 is provided on a distal end side of the universal cable 52. The connector device 54 is connected to the endoscope processor device 14. The endoscope processor device 14 includes a light source device 15 and an image processing device 16. The light source device 15 includes a processor-side connector 15A to which the connector device 54 is connected. The display 18 that displays an image image-processed by the image processing device 16 is connected to the image processing device 16. The endoscope system 12 includes a configuration in which electric power, an optical signal, and the like are transmitted in a non-contact manner between the endoscope 10 and the endoscope processor device 14 via a connector portion constituted by the connector device 54 and the processor-side connector 15A. Thus, light from the light source device 15 is transmitted via an optical fiber cable (not shown), and is applied from an illumination window 74 (see FIG. 2) provided in a distal end surface of the distal end portion 30. Further, an optical signal of an image taken in from an observation window 76 (see FIG. 2) is image-processed by the image processing device 16 and displayed as an image on the display 18.

Further, an air/water supply button 57 and a suction button 59 are provided side by side at the operating-unit main body 46. When the air/water supply button 57 is operated, air and water are supplied to the air/water supply tube 42 in FIG. 2, and the air and water can be ejected from an air/water supply nozzle 58 provided at the distal-end-portion main body 32. The air/water supply button 57 in FIG. 1 is a two-step operation button. Air is supplied to the air/water supply tube 42 by a one-step operation. Water is supplied to the air/water supply tube 42 by a two-step operation.

When the suction button 59 in FIG. 1 is operated, a body fluid such as blood can be sucked from a treatment-tool lead-out port 60 provided at the distal-end-portion main body 32 in FIG. 2 via a treatment tool channel (not shown).

As shown in FIG. 1, a pair of angle knobs 62 and 62 for bending the bending portion 28 are disposed at the operating-unit main body 46. The pair of angle knobs 62 and 62 are coaxially rotatably provided.

The raising operating lever 20 is rotatably provided coaxially with the angle knobs 62 and 62. The raising operating lever 20 is rotationally operated by a hand of an operator grasping the grasping portion 48. When the raising operating lever 20 is rotationally operated, the stranded wire 40 in FIG. 2 is pushed and pulled in accordance with the operation of the raising operating lever 20. By such an operation of the stranded wire 40, the posture of the raising base 36 connected to a distal end side of the stranded wire 40 is changed between the lowered position shown in FIG. 2 and the raised position (not shown).

As shown in FIG. 1, the grasping portion 48 of the operating unit 22 includes a treatment-tool introduction port 64 into which a treatment tool is introduced. A treatment tool (not shown) introduced from the treatment-tool introduction port 64 with a distal end portion as a leading portion is inserted into a treatment tool channel (not shown) and is led out to the outside from the treatment-tool lead-out port 60 provided in the distal-end-portion main body 32.

As shown in FIG. 1, the flexible portion 26 of the insertion unit 24 has a spiral tube (not shown) formed by spirally winding a thin strip-shaped metal plate having elasticity. The flexible portion 26 is constituted by covering the outside of the spiral tube with a cylindrical net body knitted with metal wires and covering an outer peripheral surface of the net body with an outer skin made of resin.

The bending portion 28 of the insertion unit 24 has a structure in which a plurality of angle rings (not shown) are rotatably connected to each other. The bending portion 28 is constituted by covering an outer periphery of the structure with a cylindrical net body knitted with metal wires and covering an outer peripheral surface of the net body with a cylindrical outer skin made of rubber. For example, four angle wires (not shown) are disposed from the bending portion 28 constituted as described above to the angle knobs 62 and 62, and the bending portion 28 is bent upward, downward, leftward, and rightward by pushing and pulling the angle wires by rotationally operating the angle knobs 62 and 62.

The endoscope 10 of the embodiment is, for example, a side-viewing endoscope used as a duodenoscope, and the insertion unit 24 is inserted into a subject via the oral cavity. The insertion unit 24 is inserted from the esophagus to the duodenum via the stomach, and a procedure such as a predetermined examination or treatment is performed.

Examples of treatment tools used in the endoscope 10 of the embodiment can be biopsy forceps having a cup capable of collecting a biological tissue at a distal end portion thereof, knives for EST (Endoscopic Sphincterotomy), or contrast tubes.

Next, the structure of the distal end portion 30 will be described with reference to FIGS. 2, 3, and 4.

As shown in FIG. 2, the distal end portion 30 includes the distal-end-portion main body 32 and the cap 34 attachably and detachably mounted on the distal-end-portion main body 32. As shown in FIG. 3, the distal-end-portion main body 32 has a partition wall 68 protruding toward the Y(+) direction. When the cap 34 is mounted on the distal-end-portion main body 32, a raising-base housing space 66 is formed by the partition wall 68 of the distal-end-portion main body 32 and a wall portion 34B of the cap 34. The raising-base housing space 66 is disposed at a position in the X(+) direction of the partition wall 68 and in the Y(+) direction of the treatment-tool lead-out port 60. The distal-end-portion main body 32 is made of a metal material having corrosion resistance.

As shown in FIGS. 2 and 3, the illumination window 74 and the observation window 76 are disposed adjacent to each other in the Y direction on a top surface 68A on a Z(+) side of the partition wall 68. The observation window 76 enables observation of an angle-of-view area in the Z(+) direction in which the raising-base housing space 66 opens.

The air/water supply nozzle 58 is provided at the distal-end-portion main body 32 toward the observation window 76. The observation window 76 is cleaned by air and water injected from the air/water supply nozzle 58.

As shown in FIG. 3, the partition wall 68 includes an optical-system housing chamber 72 therein. The optical-system housing chamber 72 houses an illumination unit (not shown) and an imaging unit (not shown). The illumination unit includes an illumination lens (not shown) disposed on the optical-system housing chamber 72 side of the illumination window 74, and a light guide (not shown) disposed such that a distal end surface faces the illumination lens. The light guide is disposed at the universal cable 52 from the insertion unit 24 of the endoscope 10 (see FIG. 1) via the operating unit 22. A proximal end of the light guide is connected to the connector device 54. When the connector device 54 is connected to the light source device 15, illumination light from the light source device 15 is transmitted to the illumination lens via the light guide. The illumination light is applied from the illumination window 74 to an angle-of-view area existing in the Z(+) direction.

The imaging unit includes an imaging optical system (not shown) disposed inside the observation window 76 and an imaging element (not shown) of a CMOS (complementary metal oxide semiconductor) type or a CCD (charge coupled device) type. A distal end of a signal cable (not shown) is connected to the imaging element. The signal cable is disposed at the universal cable 52 from the insertion unit 24 of the endoscope 10 (see FIG. 1) via the operating unit 22. A proximal end of the signal cable is connected to the connector device 54. When the connector device 54 is connected to the endoscope processor device 14, an imaging signal of a subject image obtained by the imaging unit is transmitted to the endoscope processor device 14 via the signal cable. The imaging signal is subjected to image processing by the endoscope processor device 14, and then displayed as a subject image on the display 18.

The distal-end-portion main body 32 is provided with a through hole 61 for inserting the stranded wire 40 (not shown).

As shown in FIG. 4, the cap 34 includes the wall portion 34B formed in a substantially cylindrical shape and having a sealed distal end side. In a part of an outer peripheral surface of the cap 34, a substantially rectangular opening window 34A is defined by the wall portion 34B. A bearing 34C extending in the Y(+) direction is formed inside the cap 34. The bearing 34C has a plate shape having a height in the Z(+) direction. The cap 34 is made of an elastic material, for example, a rubber material such as fluororubber or silicone rubber, or a resin material such as polysulfone or polycarbonate.

A rotary shaft 36B of the raising base 36 is supported in a through hole (not shown) of the bearing 34C. The rotary shaft 36B is a rod-shaped member having a length in the X-axis direction orthogonal to the bearing 34C. The rotary shaft 36B is integrally molded with the raising base 36 when molding the raising base 36. The rotary shaft 36B may be installed by providing a through hole (not shown) in the raising base 36 and inserting the rod-shaped member into the through hole.

The stranded wire 40 is directly connected and attached to the raising base 36, as will be described later. The stranded wire 40 is attached at a position adjacent to the treatment-tool guide surface 36A on a distal end side of the raising base 36.

In the present embodiment, the raising base 36 is attached to the cap 34 shown in FIG. 4, and the cap 34 with the raising base 36 is used as one component as a whole. Note that the stranded wire 40 is connected to the raising base 36.

Note that the opening window 34A of the cap 34 opens toward the Z(+) direction. That is, the opening direction of the opening window 34A of the cap 34 is orthogonal to the direction of the longitudinal axis Ax of the insertion unit and orthogonal to the axial direction (X direction) of the rotary shaft 36B.

When a procedure with the endoscope 10 ends, the cap 34, including the stranded wire 40 and the raising base 36, is removed from the distal-end-portion main body 32 and is disposed of, for example, as a disposable.

When the cap 34 is mounted on the distal-end-portion main body 32, as shown in FIG. 2, the cap 34 forms the raising-base housing space 66, and the opening window 34A opens toward the Z(+) direction. The treatment-tool lead-out port 60 of the distal-end-portion main body 32 communicates with the opening window 34A via the raising-base housing space 66.

### Method of Molding the Raising Base

Next, a method of molding the raising base will be described. In the raising base 36 molded by a method of molding an endoscope component of the present embodiment, the raising base 36 and the stranded wire 40 are directly connected to each other, and the raising base 36 and the stranded wire 40 are integrally molded when molding the raising base 36.

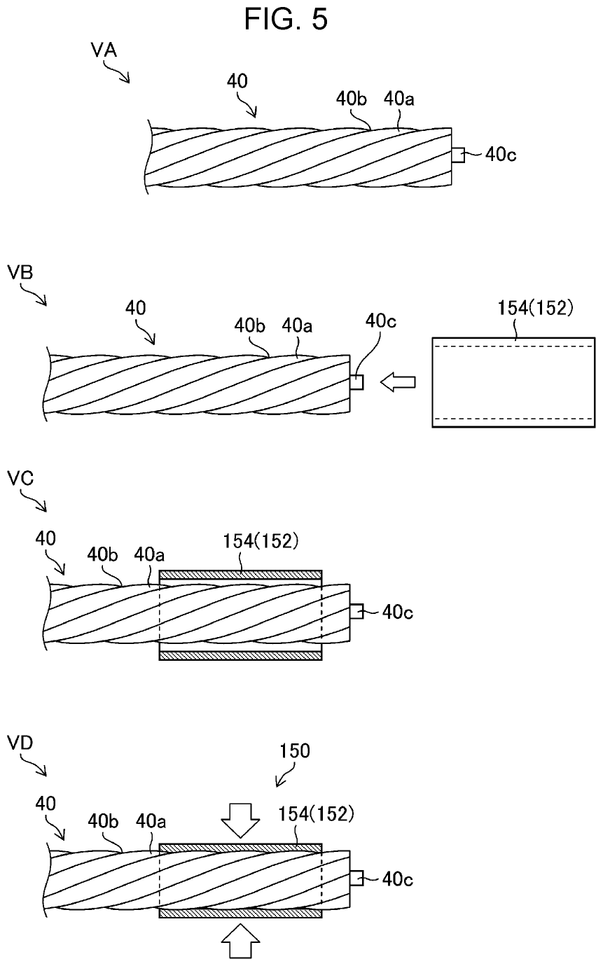

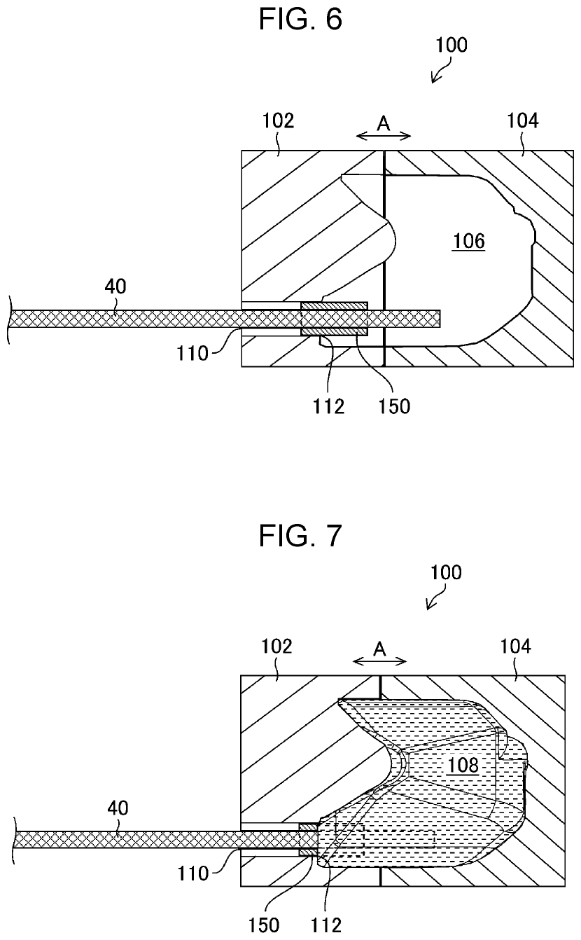

FIG. 5 is a diagram illustrating a process of forming a covering portion on a stranded wire. FIGS. 6 to 9 are diagrams illustrating the method of molding the raising base, and are plan sectional views formed by cutting at the position of the stranded wire. Note that the structure inside a cavity of a mold is omitted for simplifying the drawings.

In molding the raising base, first, as shown in FIG. 5, a covering portion 150 is formed at one end portion of the stranded wire 40. The covering portion 150 is formed by disposing a gap filling member 152 that fills gaps 40b formed between a plurality of elemental wires 40a of the stranded wire 40.

As shown by VA in FIG. 5, the stranded wire 40 is formed by twisting the plurality of elemental wires 40a around an outer peripheral portion of a core wire 40c. The stranded wire 40 is used as an operating wire for moving the raising base 36 between the lowered position and the raised position. By forming the stranded wire 40, it is possible to increase tensile strength when moving the raising base between the raised position and the lowered position, and it is possible to impart flexibility to the operating wire. In the stranded wire 40, the gaps 40b are formed between the elemental wires 40a. The covering portion 150 is formed by disposing the gap filling member 152 in the gaps 40b. In FIG. 5, a pipe 154 is used as the gap filling member 152. When the pipe 154 is used as the gap filling member 152, as shown by VB in FIG. 5, the stranded wire 40 is inserted into the pipe 154. Next, as shown by VC in FIG. 5, the pipe 154 into which the stranded wire 40 has been inserted is subjected to plastic working. As the plastic working, for example, swaging processing can be performed in which, by striking an outer peripheral surface of the pipe 154 while rotating the pipe 154, the diameter of the pipe 154 is reduced and an inner peripheral surface of the pipe 154 is brought into contact with the surface of the stranded wire 40. Further, by performing the plastic working, as shown by VD in FIG. 5, the inner peripheral surface of the pipe 154 can be brought into close contact with the surface of the stranded wire 40, and the gaps 40b of the stranded wire 40 can be filled.

Next, the method of molding the raising base 36 will be described. As shown in FIG.

6, molding of the raising base 36 is performed using a mold 100 including a first mold 102 and a second mold 104. The first mold 102 and the second mold 104 can be separated in a separation direction shown by arrow A (hereinafter, referred to as “separation direction A”). By superimposing the first mold 102 and the second mold 104 upon each other, a cavity 106 corresponding to the raising base 36 is formed inside thereof. The first mold 102 has a through hole 110 for inserting the stranded wire 40 into the cavity 106.

As the method of molding the raising base 36, as shown in FIG. 6, first, the first mold 102 and the second mold 104 are superimposed upon each other. Subsequently, a distal end side of the stranded wire 40 (a side on which the covering portion 150 is disposed) is inserted into the through hole 110 provided in the first mold 102, and the distal end side of the stranded wire 40 is inserted and disposed in the cavity 106. At this time, the stranded wire 40 is inserted and disposed such that the covering portion 150 formed on the stranded wire 40 is disposed at a boundary portion between an inner side and an outer side of the raising base 36 to be formed. In FIG. 6, a boundary portion 112 between the cavity 106 and the through hole 110 corresponds to the boundary portion between the inner side and the outer side of the raising base 36 to be formed, and the covering portion 150 is disposed at the boundary portion 112. Note that the boundary portion between the inner side and the outer side of the raising base 36 refers to a region near a surface portion of the raising base 36.

Next, as shown in FIG. 7, a formation material 108, which is a material of the raising base 36, is injected into and fills the cavity 106. A resin can be used as the formation material 108, and the cavity 106 is filled with the melted molten resin by injection molding. As the resin, for example, PEEK (Poly Ether Ketone), PC (polycarbonate), or ABS (Acrylonitrile, Butadiene, Styrene copolymer synthetic resin) can be used. By using a resin material as the formation material 108, the raising base that is to be molded can be an injection molded body made of the resin material. In addition, it is possible to, by MIM (Metal Injection Molding), fill the cavity 106 with a formation material obtained by mixing metal powder with a binder, such as a resin, and perform molding. In the case of metal injection molding, after the raising base 36 has been removed from the mold 100, degreasing is performed by heating or with a solvent, and sintering is performed to form the raising base 36. By molding the raising base by MIM, the raising base that is molded can be a metal injection molded body made of a metal material.

When PEEK is used as the formation material of the raising base 36, since the molding temperature of the raising base 36 is greater than or equal to 350° C. and less than or equal to 400° C., stainless steel (melting point: about 1400° C.) is preferably used as the material of the stranded wire 40. When a metal is used as the formation material, since the molding temperature of the raising base 36 is greater than or equal to 1200° C. and less than or equal to 1400° C., tungsten (melting point: about 3400° C.) is preferably used as the material of the stranded wire 40.

After the cavity 106 has been filled with the formation material 108, as a result of cooling, the formation material 108 is solidified to form the raising base 36 with the stranded wire 40 disposed inside the raising base 36.

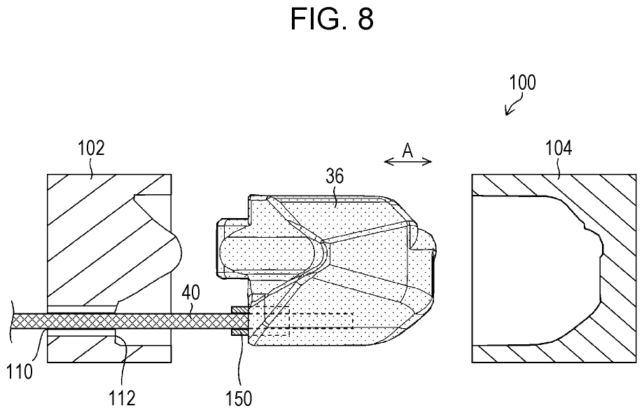

After molding the raising base 36, as shown in FIG. 8, the first mold 102 and the second mold 104 are separated in the separation direction A, and the raising base 36 is extracted from the mold 100. By pulling out the raising base 36 in the separation direction A of the mold 100, the stranded wire 40 can also be pulled out of the mold 100.

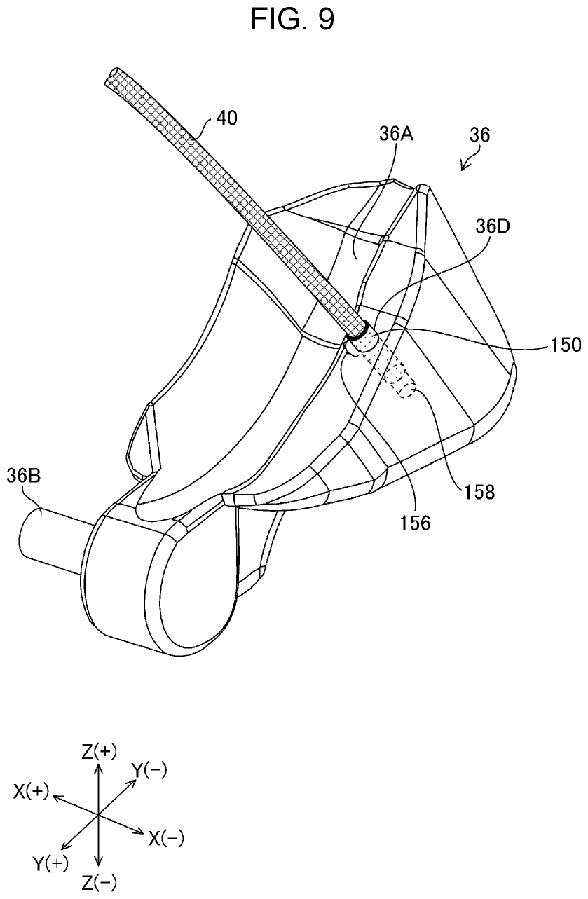

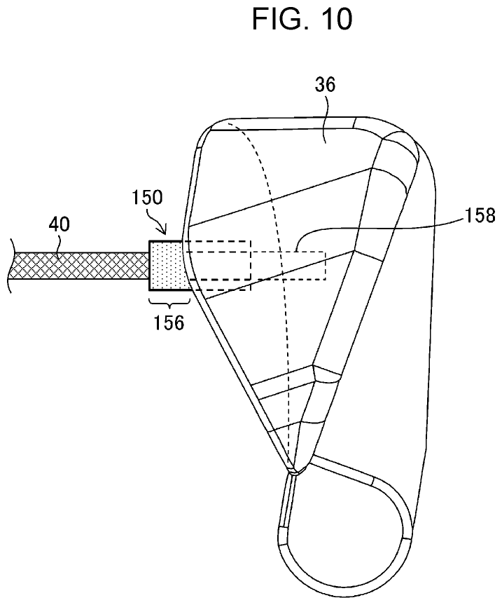

FIG. 9 is a perspective view of the raising base formed in this manner, and FIG. 10 is a side view of the raising base. As shown in FIG. 9, the raising base 36 and the stranded wire 40 are directly connected to each other at a connection portion 36D provided at a side portion of the treatment-tool guide surface 36A. Further, the covering portion 150 formed on the stranded wire 40 has an exposed portion 156 in which a part is exposed from the inside of the raising base 36 to a tension portion of the stranded wire 40.

When the cavity 106 of the mold 100 is filled with the formation material 108 and the formation material 108 is integrally molded, as a result of disposing the covering portion 150 at the boundary portion between the inner side and the outer side of the raising base 36 (the region near the surface portion of the raising base 36), it is possible to prevent the formation material 108 with which the cavity 106 is filled from flowing out of the cavity 106 from the gaps 40b between the elemental wires 40a of the stranded wire 40. In addition, by providing the covering portion 150 with the exposed portion 156, when the stranded wire 40 is inserted and disposed in the cavity 106, it is possible to prevent the formation material 108 from flowing out without strictly disposing an end portion of the covering portion 150 at the boundary portion between the inner side and the outer side of the raising base 36.

In the above-described raising base 36, an extending portion 158 exposed from the covering portion 150 is provided on a distal end side of the stranded wire 40. By providing the extending portion 158, it is not necessary to align the position of a distal end side of the covering portion 150 with the position of the distal end of the stranded wire 40. Therefore, it is not necessary to perform positioning between the covering portion 150 and the stranded wire 40, and it is possible to easily manufacture the raising base 36. However, the present invention is not limited to the configuration in which the extending portion 158 is provided, and the distal end of the stranded wire 40 and the distal end of the covering portion 150 may be located at the same position without providing the extending portion 158.

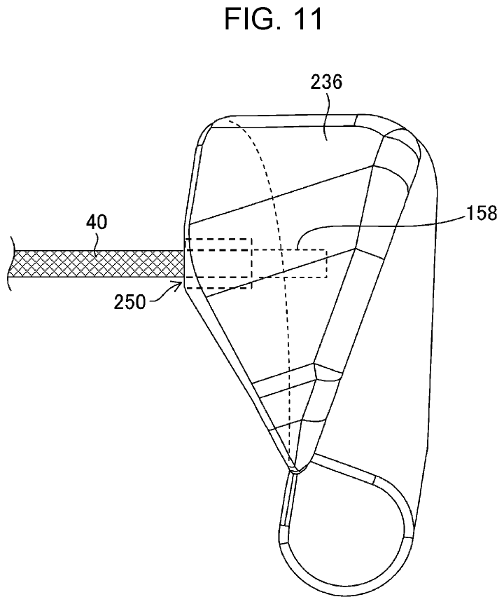

FIG. 11 is a diagram showing a modification of a molded raising base. A raising base 236 of a modification is different from the raising base 36 shown in FIGS. 9 and 10 in that a covering portion 250 is provided only inside the raising base 236 on an outer surface of a stranded wire 40. In the case of molding such a raising base 236, when the stranded wire 40 is inserted and disposed in the cavity 106, the covering portion 250 is disposed in the cavity 106, and an end portion on a proximal end side of the covering portion 250 is aligned with a boundary portion between an inner side and an outer side of the raising base 236. Even with such a configuration, the formation material 108 can be prevented from flowing out along the gaps of the stranded wire 40 as long as the elemental wires 40a of the stranded wire 40 and the formation material 108 do not come into contact with each other when molding the raising base 236.

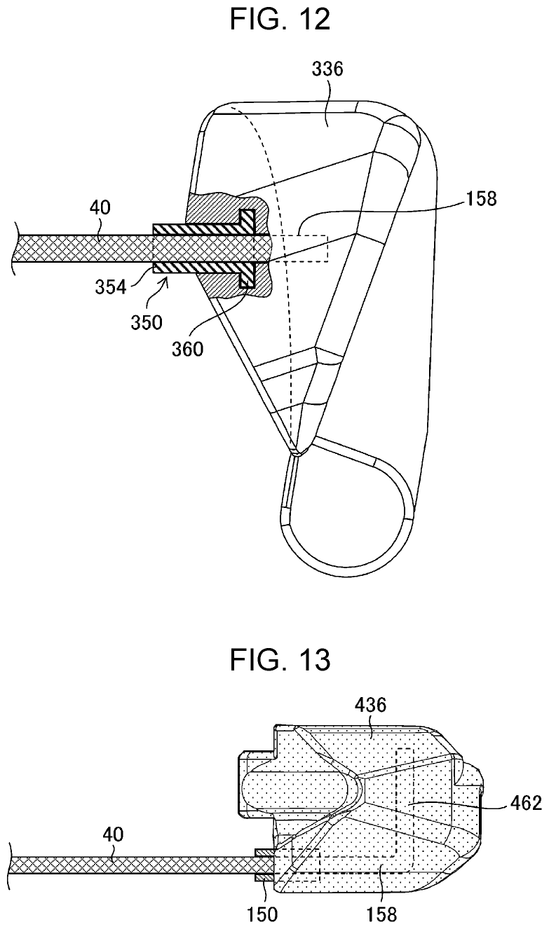

FIG. 12 is a diagram showing another modification of a raising base. In FIG. 12, a connection portion between a raising base 336 and the stranded wire 40 is illustrated in a cross-sectional view. In the raising base 336 shown in FIG. 12, a pipe 354 forming a covering portion 350 has a stepped portion 360 on a distal end side of the stranded wire 40, the stepped portion 360 having an outer diameter larger than the outer diameter of a proximal end side. By providing the stepped portion 360 on the distal end side of the stranded wire 40, in the raising base 336 in which the stranded wire 40 and the raising base 336 are integrally molded, the stepped portion 360 serves as a locking portion, and it is possible to make it difficult for the stranded wire 40 to come off the raising base 336. Thus, the tensile strength of the stranded wire 40 can be improved.

FIG. 13 is a diagram showing still another modification of a raising base. A raising base 436 shown in FIG. 13 has a bent portion 462 where the distal end of the stranded wire 40 is bent on the distal end side. By providing the bent portion 462, in the raising base 436 in which the stranded wire 40 and the raising base 436 are integrally molded, the bent portion 462 serves as a locking portion, and it is possible to make it difficult for the stranded wire 40 to come off the raising base 436. Thus, the tensile strength of the stranded wire 40 can be improved. Note that although, in FIG. 13, the bent portion 462 is provided at the extending portion 158, the bent portion 462 may be formed by bending the covering portion 150.

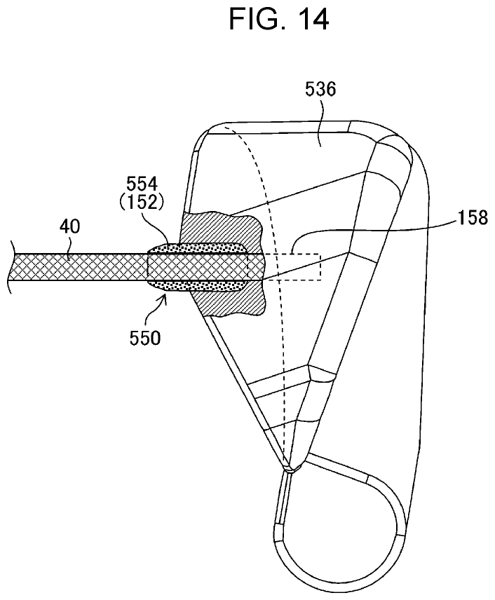

Further, although the pipe 154 has been described as an example of the gap filling member 152, any member may be used as long as it can fill the gaps 40b of the stranded wire 40. For example, as in a raising base 536 shown in FIG. 14, a covering portion 550 is formed by applying an adhesive 554 as the gap filling member 152 to the surface of the stranded wire 40, thereby making it possible to fill the gaps 40b of the stranded wire 40. Thus, the formation material 108 can be prevented from flowing out along the stranded wire 40 at the time of molding the raising base. As the adhesive 554, an adhesive having a melting point higher than that of the formation material is used. For example, when PC (melting point: 150° C.) or ABS (melting point: 100 to 125° C.) is used as the formation material of the raising base 536, an epoxy adhesive (melting point: 250 to 350° C.) having high heat resistance is used. Accordingly, it is possible to prevent the adhesive from melting at the time of molding the raising base, and to prevent the adhesive from flowing out together with the formation material. Note that, in FIG. 14, a connection portion between a raising base 536 and the stranded wire 40 is illustrated in a cross-sectional view.

The gaps 40b of the stranded wire 40 can be filled by coating the surface of the stranded wire 40 with resin by using the resin as the gap filling member 152. Thus, the formation material 108 can be prevented from flowing out along the stranded wire 40 at the time of molding the raising base 536. As the resin, a resin having a melting point higher than that of the formation material 108 is used. As the resin, PTFE (polytetrafluoroethylene (melting point: 327° C.) can be used. Accordingly, it is possible to prevent the resin from melting at the time of molding the raising base 536, and to prevent the resin from flowing out together with the formation material.

The gaps 40b of the stranded wire 40 can be filled by using solder as the gap filling member 152 and applying the solder to the surface of the stranded wire 40. Thus, the formation material 108 can be prevented from flowing out along the stranded wire 40 at the time of molding the raising base 536. As the solder, solder having a melting point higher than that of the formation material 108 is used. As the solder, lead-free solder (melting point: about 200° C.) can be used. Accordingly, it is possible to prevent the solder from melting at the time of molding the raising base 536, and to prevent the solder from flowing out together with the formation material 108.

The gaps 40b of the stranded wire 40 can be filled by using a brazing material as the gap filling member 152 and applying the brazing material to the surface of the stranded wire 40. Thus, the formation material 108 can be prevented from flowing out along the stranded wire 40 at the time of molding the raising base 536. As the brazing material, a brazing material having a melting point higher than that of the formation material 108 is used. Accordingly, it is possible to prevent the brazing material from melting at the time of molding the raising base 536, and to prevent the brazing material from flowing out together with the formation material 108.

Note that, in the above-described embodiment, the method of molding a raising base as an endoscope component has been described. However, the present invention is not limited to a raising base, and can be applied to a method of molding an endoscope component in which the endoscope component, which constitutes an endoscope, and a stranded wire are integrally molded. In addition to being applied to a raising base, such an endoscope component can be applied, to, for example, a bending tube portion that constitutes a bending portion and is integrally molded with a bending operating wire, an operation slide member that is integrally molded with an operating wire, and a hood that is integrally molded with a wire and moves in an axial direction by pushing and pulling the wire.

### Other Embodiments

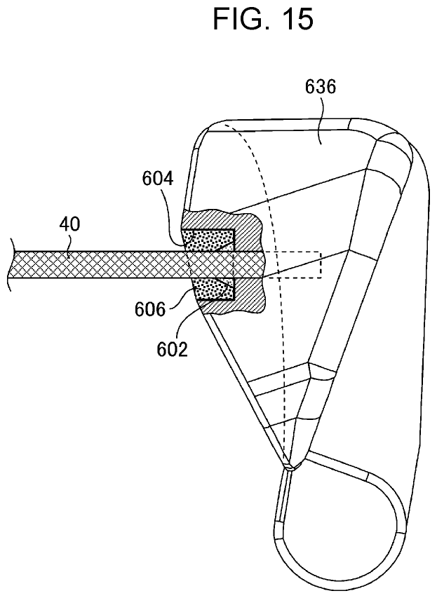

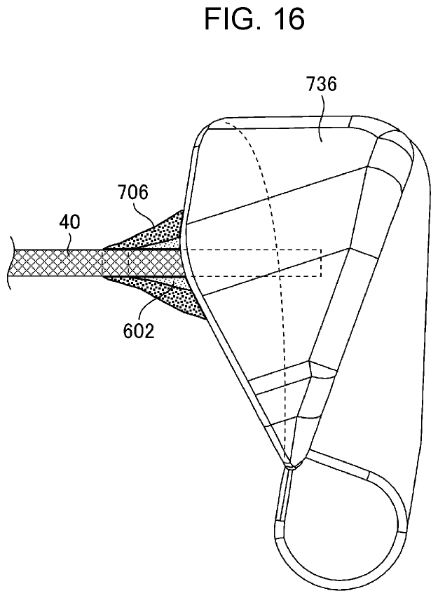

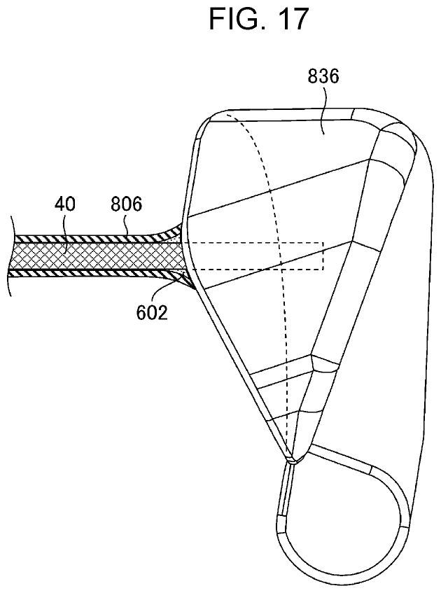

FIGS. 15 to 17 are diagrams showing embodiments for preventing a formation material that has flowed out along a stranded wire from peeling off when integrally molding a raising base and the stranded wire. The embodiments shown in FIGS. 15 to 17 include a falling-off prevention member for preventing a falling-off of an outflow portion 602 in which a formation material flows out after molding the raising base (endoscope component). Note that, in FIG. 15, a connection portion between a raising base 636 and a stranded wire 40 is illustrated in a cross-sectional view. In FIGS. 16 and 17, a connection portion of a stranded wire is illustrated in a cross-sectional view.

The raising base 636 shown in FIG. 15 has a member reservoir portion 604 at a draw-out portion of the stranded wire 40. The member reservoir portion 604 is provided so as to be recessed from the surface of the raising base and can be formed by changing the shape of a cavity of a mold. After molding the raising base, by filling the member reservoir portion 604 with a falling-off prevention member 606, the outflow portion 602 in which a formation material flows out along the stranded wire 40 can be covered. Accordingly, it is possible to prevent the outflow portion 602 from falling off.

In addition, in FIG. 16, a member reservoir portion is not provided, and a falling-off prevention member 706 is disposed so as to cover the outflow portion 602 that has flowed out when molding a raising base 736, thereby making it is possible to prevent the outflow portion 602 that has flowed out along the stranded wire 40 from falling off.

In the embodiments shown in FIGS. 15 and 16, an adhesive can be used as the falling-off prevention members 606 and 706. In addition to the adhesive, by applying a resin, solder, or a brazing material, it is possible to prevent a solidified formation material from falling off.

The raising base shown in FIG. 17 uses a contractible tube as a falling-off prevention member 806. By disposing a contractible tube from an outer surface of the outflow portion 602 that has flowed out when molding a raising base 836, it is possible to prevent, due to the contraction of the tube, the tube from coming into close contact with the outflow portion 602, and the outflow portion 602 from falling off.

According to the present embodiment, even when a formation material flows out from the inside of a cavity of a mold and is solidified, by disposing the falling-off prevention member, it is possible to prevent a falling-off of the outflow portion 602 in which the formation material flows out and is solidified at the time of molding the raising base.

In the endoscopes of these embodiments, an endoscope component (a raising base in each of the present embodiments) constituting the endoscope is integrally molded with a stranded wire composed of a plurality of elemental wires. The method of molding such an endoscope component has a step of inserting and disposing one end portion of a stranded wire into a cavity of a mold; a step of injecting a formation material, which is a material of the endoscope component, into the cavity and filling the cavity with the formation material to integrally mold the endoscope component and the stranded wire; a step of removing the endoscope component integrally molded with the stranded wire from the mold; and a step of disposing a falling-off prevention member on the formation material that has flowed out along the gaps of the stranded wire from the inside of the cavity.

Further, the endoscope molded in this way is an endoscope including an endoscope component, the endoscope component being an integrally molded body integrally molded with a stranded wire composed of a plurality of elemental wires, and is an endoscope including a falling-off prevention member at a part exposed from the endoscope component at a draw-out portion of the stranded wire.

### REFERENCE SIGNS LIST

10 endoscope

12 endoscope system

14 endoscope processor device

15 light source device

15A processor side connector

16 image processing apparatus

18 display

20 raising operating lever

22 operating unit

24 insertion unit

26 flexible portion

28 bending portion

30 distal end portion

32 distal-end-portion main body

34 cap

34A opening window

34B wall portion

34C bearing

36, 236, 336, 436, 536, 636, 736, 836 raising base

36A treatment-tool guide surface

36B rotary shaft

36D connection portion

40 stranded wire

40a elemental wire

40b gap

40c core wire

42 air/water supply tube

44 cable insertion channel

45 insertion channel

46 operating-unit main body

48 grasping portion

50 bend prevention tube

52 universal cable

54 connector device

57 air/water supply button

58 air/water supply nozzle

59 suction button

60 treatment-tool lead-out port

61 through hole

62 angle knob

64 treatment-tool introduction port

66 raising-base housing space

68 partition wall

68A top surface

72 optical-system housing chamber

74 illumination window

76 observation window

100 mold

102 first mold

104 second mold

106 cavity

108 formation material

110 through hole

112 boundary portion

150, 250, 350, 550 covering portion

152 gap filling member

154, 354 pipe

156 exposed portion

158 extending portion

360 stepped portion

462 bent portion

554 adhesive

602 outflow portion

604 member reservoir portion

606, 706, 806 falling-off prevention member

What is claimed is:

1. A method of molding an endoscope component, in which the endoscope component constituting an endoscope is integrally molded with a stranded wire composed of a plurality of elemental wires, the method comprising:
a step of forming a covering portion, in which a gap filling member that fills a gap formed between the plurality of elemental wires is disposed, at one end portion of the stranded wire;
a step of inserting and disposing the one end portion of the stranded wire into a cavity of a mold such that the covering portion is disposed at a boundary portion between an inner side and an outer side of the endoscope component;
a step of injecting a formation material, which is a material of the endoscope component, into the cavity and filling the cavity with the formation material to integrally mold the endoscope component and the stranded wire; and
a step of removing the endoscope component integrally molded with the stranded wire from the mold.

2. The method of molding an endoscope component according to claim 1, wherein, in the inserting and disposing step, a part of the covering portion is disposed on an outer side of the cavity.

3. The method of molding an endoscope component according to claim 1, wherein the endoscope component is formed by injection molding of a resin.

4. The method of molding an endoscope component according to claim 1, wherein the endoscope component is formed by metal injection molding of metal powder.

5. The method of molding an endoscope component according to claim 1, wherein, in the step of forming the covering portion, the gap of the stranded wire is filled by applying an adhesive having a melting point higher than a melting point of the formation material to the stranded wire.

6. The method of molding an endoscope component according to claim 1, wherein, in the step of forming the covering portion, the gap of the stranded wire is filled by coating the stranded wire with a resin having a melting point higher than a melting point of the formation material.

7. The method of molding an endoscope component according to claim 1, wherein, in the step of forming the covering portion, the gap of the stranded wire is filled by applying solder having a melting point higher than a melting point of the formation material to the stranded wire.

8. The method of molding an endoscope component according to claim 1, wherein, in the step of forming the covering portion, the gap of the stranded wire is filled by applying a brazing material having a melting point higher than a melting point of the formation material to the stranded wire.

9. The method of molding an endoscope component according to claim 1, wherein, in the step of forming the covering portion, the gap of the stranded wire is filled by subjecting a pipe into which the stranded wire has been inserted to plastic working.

10. The method of molding an endoscope component according to claim 1, wherein the endoscope component is a raising base disposed at a distal-end-portion main body provided on a distal end side of an insertion unit of the endoscope.

11. An endoscope comprising:
an endoscope component,
wherein the endoscope component is an integrally molded body integrally molded with a stranded wire composed of a plurality of elemental wires,
wherein the stranded wire has a covering portion, in which a gap filling member that fills a gap formed between the plurality of elemental wires is disposed, at one end portion of the stranded wire; and
wherein the covering portion is disposed at a draw-out portion of the stranded wire inside the endoscope component.

12. The endoscope according to claim 11, wherein the covering portion comprises an exposed portion exposed from the endoscope component.

13. The endoscope according to claim 11, wherein the stranded wire comprises an extending portion on a distal end side of the stranded wire, the extending portion being exposed from the covering portion in the endoscope component.

14. The endoscope according to claim 11, wherein the stranded wire has a bent portion that is bent on a distal end side of the stranded wire in the endoscope component.

15. The endoscope according to claim 11, wherein the endoscope component is an injection molded body made of a resin material.

16. The endoscope according to claim 11, wherein the endoscope component is a metal injection molded body made of a metal material.

17. The endoscope according to claim 11, wherein the gap filling member is an adhesive having a melting point higher than a melting point of a material constituting the endoscope component, and is provided in the gap of the stranded wire.

18. The endoscope according to claim 11, wherein the gap filling member is a resin having a melting point higher than a melting point of a material constituting the endoscope component, and is provided in the gap of the stranded wire.

19. The endoscope according to claim 11, wherein the gap filling member is solder having a melting point higher than a melting point of a material constituting the endoscope component, and is provided in the gap of the stranded wire.

20. The endoscope according to claim 11, wherein the gap filling member is a brazing material having a melting point higher than a melting point of a material constituting the endoscope component, and is provided in the gap of the stranded wire.

21. The endoscope according to claim 11, wherein the gap filling member is a pipe externally fitted to an outer peripheral surface of the stranded wire.

22. The endoscope according to claim 21, wherein the covering portion has a stepped portion on a distal end side of the stranded wire, the stepped portion having an outer diameter larger than an outer diameter of a proximal end side.

23. The endoscope according to claim 11, wherein the endoscope component is a raising base disposed at a distal-end-portion main body provided on a distal end side of an insertion unit of the endoscope.
